(19) United States
(12) Patent Application Publication
(10) Pub. No.: US 2006/0161244 A1
(43) Pub. Date: Jul. 20, 2006

(54) Vascular graft and deployment system

(76) Inventor: Jacques Seguin, Old Windsor (GB)

Correspondence Address: KNOBBE MARTENS OLSON & BEAR LLP, 2040 MAIN STREET, FOURTEENTH FLOOR, IRVINE, CA, 92614, US

(21) Appl. No.: 11/266,017

(22) Filed: Nov. 3, 2005

Related U.S. Application Data
(63) Continuation-in-part of application No. 10/836,317, filed on Apr. 30, 2004.
(60) Provisional application No. 60/624,692, filed on Nov. 3, 2004.
(60) Provisional application No. 60/467,625, filed on May 2, 2003.

(51) Int. Cl. A61F 2/06

(52) U.S. Cl. 623/1.23; 623/1.35; 623/1.16; 623/1.36

(57) ABSTRACT

Disclosed is a method and apparatus for treating bifurcations of the vascular system, such as abdominal aneurysms at the bifurcation of the aorta and iliac arteries. A tubular implant having a first section, a second section and a magnetic connection therebetween is positioned across the bifurcation such that the proximal ends of the first and second sections extends into a first iliac and a second iliac respectively. Deployment catheters are also disclosed.

### PRIORITY INFORMATION

This application claims the benefit under 35 USC §119(e) of U.S. Provisional Application No. 60/624,692, filed Nov. 3, 2005 and is a continuation-in-part of application Ser. No. 10/836,317, filed Apr. 30, 2004, claims the benefit under 35 USC §119(e) of U.S. Provisional Application No. 60/467,625, filed May 2, 2003.

### BACKGROUND OF THE INVENTION

1. Field of the Invention

The present invention relates to vascular grafts and vascular graft deployment systems.

2. Description of the Related Art

An abdominal aortic aneurysm is a sac caused by an abnormal dilation of the wall of the aorta, a major artery of the body, as it passes through the abdomen. The abdomen is that portion of the body which lies between the thorax and the pelvis. It contains a cavity, known as the abdominal cavity, separated by the diaphragm from the thoracic cavity and lined with a serous membrane, the peritoneum. The aorta is the main artery, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries.

The aneurysm usually arises in the infrarenal portion of the diseased aorta, for example, below the kidneys. When left untreated, the aneurysm may eventually cause rupture of the sac with ensuing fatal hemorrhaging in a very short time. High mortality associated with the rupture led initially to transabdominal surgical repair of abdominal aortic aneurysms. Surgery involving the abdominal wall, however, is a major undertaking with associated high risks. There is considerable mortality and morbidity associated with this magnitude of surgical intervention, which in essence involves replacing the diseased and aneurysmal segment of blood vessel with a prosthetic device which typically is a synthetic tube, or graft, usually fabricated of Polyester, Urethane, DACRON™, TEFLON.™, or other suitable material.

To perform the surgical procedure requires exposure of the aorta through an abdominal incision which can extend from the rib cage to the pubis. The aorta must be closed both above and below the aneurysm, so that the aneurysm can then be opened and the thrombus, or blood clot, and arteriosclerotic debris removed. Small arterial branches from the back wall of the aorta are tied off. The DACRONTM™. tube, or graft, of approximately the same size of the normal aorta is sutured in place, thereby replacing the aneurysm. Blood flow is then reestablished through the graft. It is necessary to move the intestines in order to get to the back wall of the abdomen prior to clamping off the aorta.

If the surgery is performed prior to rupturing of the abdominal aortic aneurysm, the survival rate of treated patients is markedly higher than if the surgery is performed after the aneurysm ruptures, although the mortality rate is still quite high. If the surgery is performed prior to the aneurysm rupturing, the mortality rate is typically slightly less than 10%. Conventional surgery performed after the rupture of the aneurysm is significantly higher, one study reporting a mortality rate of 66.5%. Although abdominal aortic aneurysms can be detected from routine examinations, the patient does not experience any pain from the condition. Thus, if the patient is not receiving routine examinations, it is possible that the aneurysm will progress to the rupture stage, wherein the mortality rates are significantly higher.

Disadvantages associated with the conventional, prior art surgery, in addition to the high mortality rate include the extended recovery period associated with such surgery; difficulties in suturing the graft, or tube, to the aorta; the loss of the existing aorta wall and thrombosis to support and reinforce the graft; the unsuitability of the surgery for many patients having abdominal aortic aneurysms; and the problems associated with performing the surgery on an emergency basis after the aneurysm has ruptured. A patient can expect to spend from one to two weeks in the hospital after the surgery, a major portion of which is spent in the intensive care unit, and a convalescence period at home from two to three months, particularly if the patient has other illnesses such as heart, lung, liver, and/or kidney disease, in which case the hospital stay is also lengthened. Since the graft must be secured, or sutured, to the remaining portion of the aorta, it is many times difficult to perform the suturing step because the thrombosis present on the remaining portion of the aorta, and that remaining portion of the aorta wall may many times be friable, or easily crumbled.

Since many patients having abdominal aortic aneurysms have other chronic illnesses, such as heart, lung, liver, and/or kidney disease, coupled with the fact that many of these patients are older, the average age being approximately 67 years old, these patients are not ideal candidates for such major surgery.

More recently, a significantly less invasive clinical approach to aneurysm repair, known as endovascular grafting, has been developed. Parodi, et al. provide one of the first clinical descriptions of this therapy. Parodi, J. C., et al., “Transfemoral Intraluminal Graft Implantation for Abdominal Aortic Aneurysms,” 5 Annals of Vascular Surgery 491 (1991). Endovascular grafting involves the transluminal placement of a prosthetic arterial graft within the lumen of the artery.

In general, transluminally implantable prostheses adapted for use in the abdominal aorta comprise a tubular wire cage surrounded by a tubular PTFE or Dacron sleeve. Both balloon expandable and self expandable support structures have been proposed. Endovascular grafts adapted to treat both straight segment and bifurcation aneurysms have also been proposed. For bifurcated aneurysms, it has been suggested that the prosthesis be formed from two separate parts. In such systems, the first part may extend from the aorta into the first iliac branch. The second part is for the second iliac branch. The two parts are linked together during surgery. This complicates the surgical procedure and makes it more time consuming. In addition, the connection between the two parts may leak and cause blood to enter the aneurysm. Furthermore, because the first part of the prosthesis is designed for the aorta, it requires a relatively large delivery system (e.g., 6 to 8 millimeters or 18-24 French) to delivery the compressed prosthesis. Such a large delivery system may require surgical cut-down to enter the vessel lumen.

Notwithstanding the foregoing, there remains a need for a structurally simple, easily deployable transluminally implantable endovascular prosthesis.

### SUMMARY OF THE INVENTION

In one embodiment of the present invention, a self expandable bifurcation graft, comprises a first tubular body, having a superior end and an inferior end, and a second tubular body, having a superior end and an inferior end. A magnetic connection is provided between the superior end of the first tubular body and the superior end of the second tubular body. The superior ends of the first and second tubular bodies are configured such that when the tubular bodies are connected by the magnetic connection about into a side by side relationship, each of the superior ends defines a semi circular opening.

Another embodiment of the present invention comprises a method of deploying a prosthesis. In the method, a deployment apparatus is provided and comprises an first outer sheath having a device distal end and a device proximal end and a second outer sheath also having a device distal end and a device proximal end. A vascular prosthesis is also provided and comprises first and second tubular segments. The first tubular segment is positioned within the first outer sheath and the second tubular segment is positioned in the second outer sheath. The first and second outer sheaths are advanced independently through an ipsilateral ialac artery and contralateral iliac artery in a distal direction until the distal ends of the first and second outer sheaths are positioned at an aortic neck. The first and second outer sheaths are proximally retracted to deploy the prosthesis and allow the distal ends of the first and second tubular segments to attach to each other.

Another embodiment of the present invention comprises a method of treating an aneurysm near the bifurcation of a vessel into a first branch and a second branch. In the method, a first prosthesis is positioned at a position proximal, with respect to blood flow, to the aneurysm. A catheter is provided and has a device proximal portion, a device distal portion, and a deployment zone therebetween. The catheter is positioned such that the device proximal portion extends into the first branch, the device distal portion extends into the second branch, and the deployment zone is aligned with the vessel. The deployment zone is advanced superiorly into the vessel. A bifurcation graft is advanced from the catheter such that a device distal end of the bifurcated graft is positioned within the first prosthesis.

Another embodiment of the present invention comprises a vascular prosthesis assembly that includes a first prosthesis comprising a tubular structure and a second prosthesis. The second prosthesis comprises a first tubular segment having a device distal end and a device proximal end, the distal end defining a distal opening and the proximal end defining a proximal opening. The second prosthesis also comprises a second tubular segment also having a device distal end and a device proximal end, the distal end defining a distal opening and the proximal end defining a proximal opening. A flexible link connects the distal ends of the first and second tubular segments. The device distal ends of the first and second tubular segments are configured to be expanded within the first prosthesis.

Another embodiment of the present invention comprises a vascular prosthesis that includes a first tubular segment having a device distal end and a device proximal end, the distal end defining a distal opening and the proximal end defining a proximal opening. The first tubular segment comprises a polymeric sleeve and a support structure comprising a series of end to end segments, each segment comprising a zig-zag frame. The prosthesis also includes a second tubular segment also having a device distal end and a device proximal end, the distal end defining a distal opening and the proximal end defining a proximal opening. The second tubular segment comprises a polymeric sleeve and a support structure comprising a series of end to end segments, each segment comprising a zig-zag frame. A flexible link connects the distal ends of the first and second tubular segments. At the device distal ends of the first and second tubular segments, the polymeric sleeves have a zig-zag edge that follows, at least partially, the zig-zag frame.

Another embodiment of the present invention comprises a vascular prosthesis assembly that includes a deployment apparatus and a vascular prosthesis. The deployment apparatus comprises a first outer sheath having a device distal end and a device proximal end and a second outer sheath also having a device distal end and a device proximal end. The vascular prosthesis is positioned within the first and second outer sheaths. The vascular prosthesis comprises a first and second tubular segments that are connected together at their distal ends by a link. A first pusher positioned within the first outer sheath between a device distal end of the first outer sheath and a first tubular segment of the vascular prosthesis. A second pusher is positioned within the second outer sheath between a device proximal end of the second outer sheath and a second tubular segment of the vascular prosthesis.

Further features and advantages of the present invention will become apparent to those of ordinary skill in the art in view of the detailed description of preferred embodiments which follow, when considered together with the attached drawings and claims.

### BRIEF DESCRIPTION OF THE DRAWINGS

FIG. 1 is a schematic representation of a vascular prosthesis, having certain features and advantages according to an embodiment of the invention, positioned within an abdominal aortic aneurysm.

FIG. 2 is a side perspective view of the vascular prosthesis of FIG. 1.

FIG. 3 is a top perspective view of the vascular prosthesis of FIG. 1 in a straightened configuration.

FIG. 4 is a top (anatomically proximal end) plan view of the vascular prosthesis of FIG. 1.

FIG. 5 is a side view of a modified embodiment of the vascular prosthesis of FIG. 1.

FIG. 5A is a side view of a modified embodiment of prosthesis of FIG. 1.

FIG. 6A is a partial cross-sectional view of a deployment apparatus having certain features and advantages according to an embodiment of the invention.

FIG. 6B is a partial cross-sectional view of a modified embodiment of a deployment apparatus.

FIG. 6C is a partial cross-sectional view of another modified embodiment of a deployment apparatus.

FIG. 6D is a side view of an inner member than can be used in combination with the a deployment apparatuses of FIGS. 6A-6C.

FIG. 7 is a schematic representation of a guidewire positioned across the ipsilateral and contralateral iliacs.

FIG. 8 is a schematic representation of a deployment apparatus of FIG. 6 positioned across the ipsilateral and contralateral iliacs.

FIG. 9 is a schematic representation of a deployment apparatus of FIG. 6 partially withdrawn and two guidewires positioned across the aneurysm into the aorta.

FIG. 10 is a schematic representation of a deployment apparatus of FIG. 6 positioned across the aneurysm into the aorta.

FIG. 11 is a schematic representation of a deployment apparatus of FIG. 6 positioned across the aneurysm into the aorta and partially withdrawn to deploy the vascular prosthesis.

FIG. 12 a schematic representation of a vascular prosthesis, having certain features and advantages according to another embodiment of the invention, positioned within an abdominal aortic, aneurysm.

FIG. 13 is a side view of the vascular prosthesis of FIG. 12.

FIGS. 14A and 14B are schematic representations of a vascular prosthesis, having certain features and advantages according to another embodiment of the invention, positioned within an abdominal aortic aneurysm.

FIG. 15 is a schematic representation of a mechanical linkage that may be used with the prosthesis of FIG. 14.

FIG. 16 is a schematic representation of a deployment apparatus and two guidewires positioned across the aneurysm into the aorta.

FIG. 17 is a schematic representation of a deployment apparatus of FIG. 16 positioned across the aneurysm into the aorta.

FIG. 18 is a schematic representation of a deployment apparatus of FIG. 16 positioned across the aneurysm into the aorta and partially withdrawn to deploy the vascular prosthesis.

FIG. 19 is a schematic representation of a vascular prosthesis assembly according to an embodiment of the invention positioned within an abdominal aortic aneurysm.

FIG. 20 is a schematic representation of another vascular prosthesis assembly according to an embodiment of the invention positioned within an abdominal aortic aneurysm.

FIG. 21 is a side perspective view of a pusher for a deployment apparatus according to an embodiment of the invention.

FIG. 22 is a side view of a device proximal end of a prosthesis according to an embodiment of the invention.

### DETAILED DESCRIPTION OF THE PREFERRED EMBODIMENT

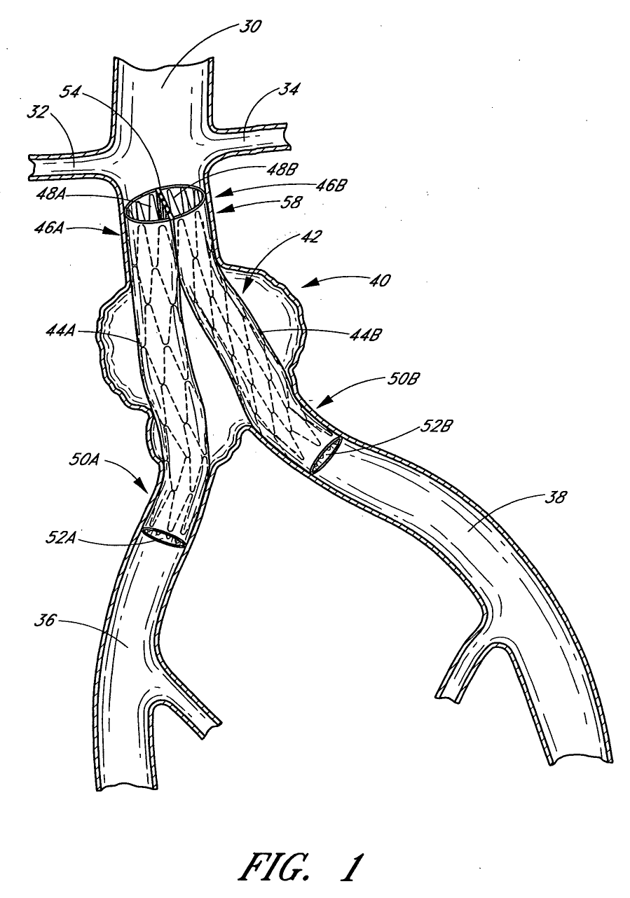

FIG. 1 illustrates a schematic representation of the abdominal part of the aorta and its principal branches. In particular, the abdominal aorta 30 is characterized by a right renal artery 32 and left renal artery 34. The large terminal branches of the aorta 30 are the right and left common iliac arteries 36 and 38. Additional vessels (e.g., second lumbar, testicular, inferior mesenteric, middle sacral) have been omitted for simplification. An aneurysm 40 is illustrated in the infrarenal portion of the diseased aorta. An endoluminal vascular prosthesis 42, in accordance with an embodiment of the present invention, is illustrated spanning the aneurysm 40.

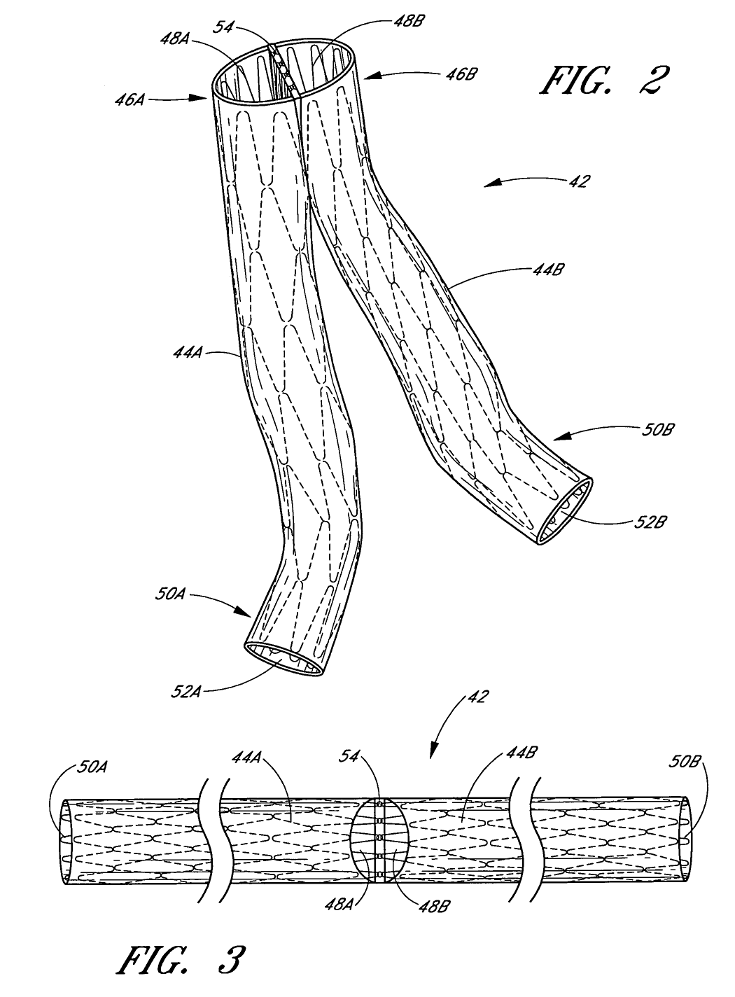

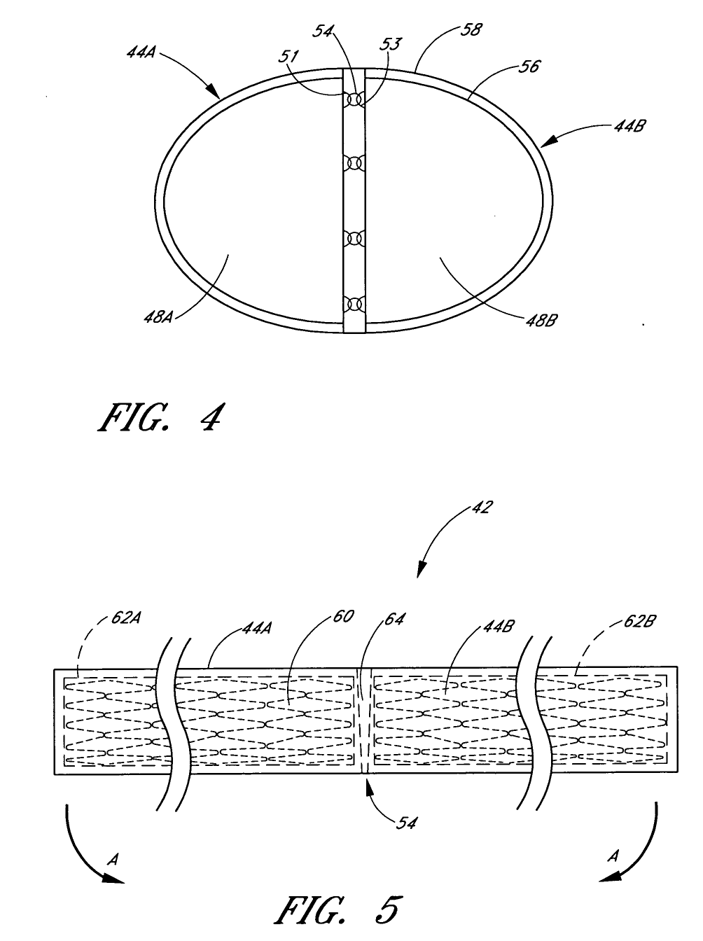

With reference to FIGS. 1-4, the prosthesis 42 comprises a first tubular member or tube 44A and a second tubular member or tube 44B. The first tubular member 44A has a device distal end 46A, which defines a device distal opening 48A, and a device proximal end 50A, which defines a proximal opening 52A. In a similar manner, the second tubular member 44B has a device proximal end 46B, which defines a proximal opening 48B, and a device distal end 50B, which defines a distal opening 52B. As best seen in FIG. 1, each tubular member 44A, 44B is adapted such that it can extend across the aneurysm 40.

As will be understood in view of the disclosure herein, the device distal end 46A of first or proximal tubular section 44A along with the device proximal end 46B of the second or distal section 44B are both implanted in the anatomically proximal (with respect to blood flow) or superior orientation. The device proximal end 50A and device distal end 50B of iliac branches 44A and 44B, as implanted, are in the anatomically distal (with respect to blood flow) or inferior position. In the description herein, the terms “distal” and “proximal” are used with reference to a user of the device being described unless a different reference point is identified (e.g., blood flow).

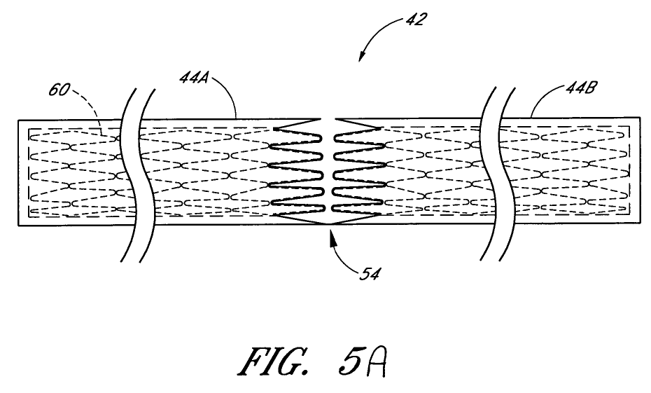

The distal end 46A and proximal end 46B of the tubes 44A, 44B are connected together by a flexible connection or hinge 54 such as a flexible material or link, which will be described in detail below. As best seen in FIG. 4, the opposing ends 46A, 46B of the tubes 44A, 44B have a generally D-shaped or other complementary cross-section such that the tubes 44A, 44B define a periphery 56 when the tubes 44A and 44B are folded into a side by side orientation, which preferably closely conforms to the cross-sectional shape of the aorta 58 at the superior end of the aneurysm 40. This arrangement advantageously seals off or isolates the aneurysm 40 from blood flow while directing the blood into the openings 48A, 48B of the first and second tubes 44A, 44B.

The flexible connection 54 defines a preferably sealed interface between the openings 48A, 48B of the tubes 44A, 44B. In the illustrated embodiment, this interface defines a generally flat side in contrast to the generally rounded shape of the periphery 56. However, in modified embodiments, the interface can be of a different shape (e.g., rounded, jagged etc.).

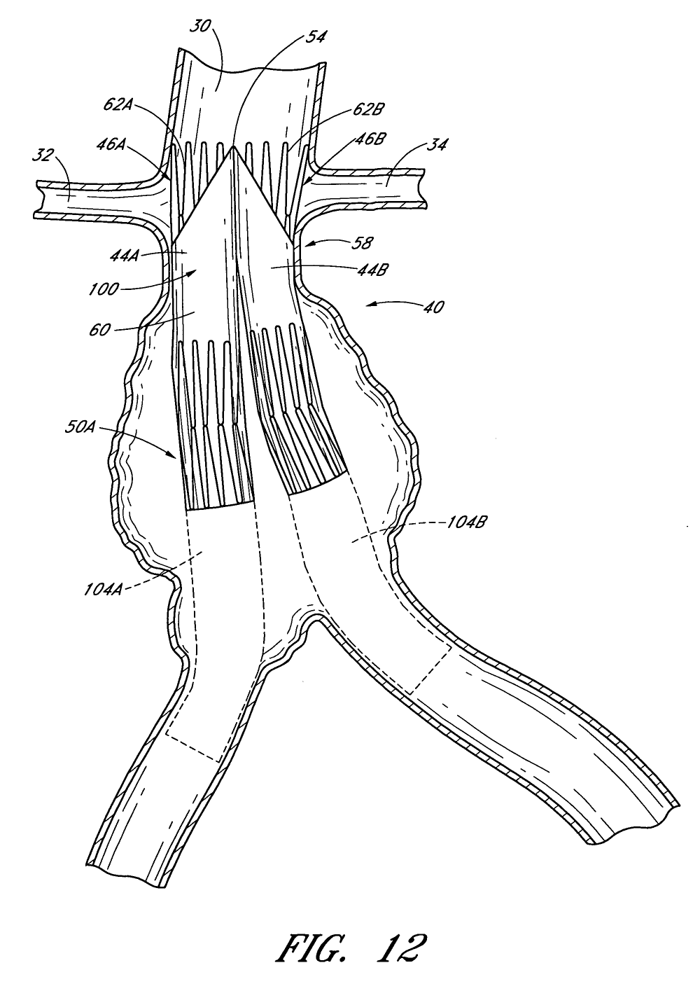

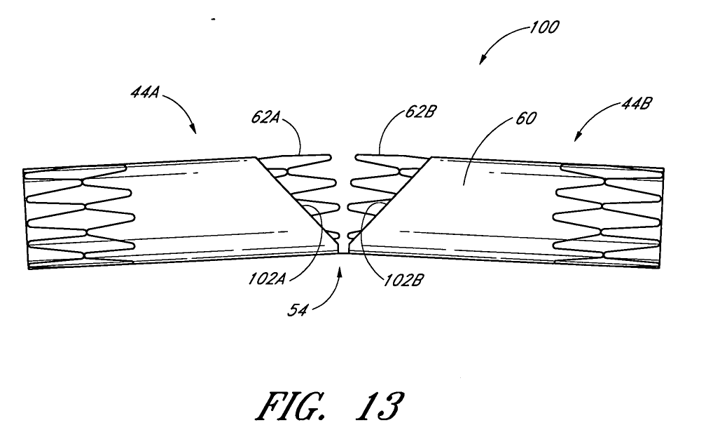

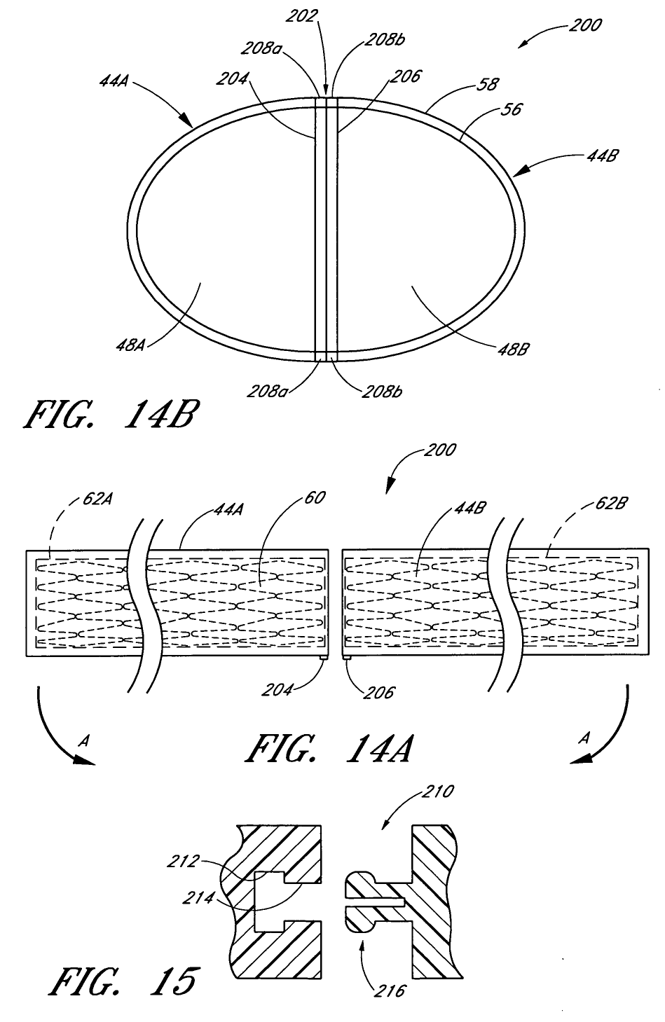

As best seen in FIG. 1, the opposing ends 46A, 46B of the prosthesis are preferably positioned as close as possible to the lowest renal artery so as to maximize the overlap between graft material and the healthy infrarenal aortic wall 58 and thereby promoting a good seal within the artery. In modified embodiments, the prosthesis may be extended over or beyond the renal arteries. In such embodiments (see e.g. FIGS. 12 and 13 discussed below), the portion of the prosthesis extending over and/or beyond the renal arteries is advantageously not covered with a graft material.

As best seen in FIG. 1, the proximal and distal openings 52A, 52B of the tubes 44A, 44B are preferably configured to closely conform to the cross-sectional shape of the right and left common iliac arteries 36, 38. The openings 52A, 52B therefore have a substantially round or O-shaped cross-section as compared to the superior openings 48A, 48B. As such, each tube 44A, 44B transitions from the generally D-shaped openings 48A, 48B at the superior end to the generally O-shaped openings 52A, 52B at the inferior end.

The vascular prosthesis 42 can be formed using a variety of known techniques. For example, in one embodiment, each tube 44A, 44B comprises an expandable tubular support or skeleton and a polymeric or fabric sleeve that is situated concentrically outside and/or inside of the tubular support. In another embodiment, the tubular support may be embedded within a polymeric matrix which makes up the sleeve. Regardless of whether the sleeve is inside or outside the support, the sleeve may be attached to the tubular support by any of a variety of techniques, including laser bonding, adhesives, clips, sutures, dipping or spraying or others, depending upon the composition of the sleeve and overall prosthesis design.

The sleeve may be formed from any of a variety of synthetic polymeric materials, or combinations thereof, including ePTFE, PE, PET, Urethane, Dacron, nylon, polyester or woven textiles. In one embodiment, the material of sleeve is sufficiently porous to permit ingrowth of endothelial cells, thereby providing more secure anchorage of the prosthesis and potentially reducing flow resistance, sheer forces, and leakage of blood around the prosthesis. Alternatively, materials that inhibit endothelial growth may also be used. Porosity in polymeric sleeve materials may be estimated by measuring water permeability as a function of hydrostatic pressure, which will preferably range from about 3 to 6 psi.

The porosity characteristics of the polymeric sleeve may be either homogeneous throughout the axial length of the prosthesis 42, or may vary according to the axial position along the prosthesis 42. For example, with reference to FIG. 1, different physical properties may be called upon at different axial positions along the prosthesis 42 in use. For example, in the illustrated embodiment, the distal ends 46A, 50B, and the proximal ends 50A, 46B of the prosthesis 42 will seat against the native vessel wall, on either side of the aneurysm 40. In these end portions, the prosthesis may be configured to encourage endothelial growth, or, to permit endothelial growth to infiltrate portions of the prosthesis in order to enhance anchoring and minimize leakage. The central portion of the prosthesis spans the aneurysm, and therefore anchoring is less of an issue. Instead, maximizing lumen diameter and minimizing blood flow through the prosthesis wall become primary objectives. Thus, the central portions of the prosthesis 42, the polymeric sleeve may either be nonporous, or provided with pores of relatively lower porosity.

In another embodiment, the ends 46A, 46B, 50A, 50B of prosthesis 42 may be provided with any of a variety of tissue anchoring structures, such as, for example, barbs, hooks, and/or exposed portions of the tubular support. Such anchoring structures over time, may become embedded in cell growth on the interior surface of the vessel wall. These configurations advantageously resist migration of the prosthesis within the vessel and reduce leakage around the ends of the prosthesis. The specific number, arrangement and/or structure of such anchoring structures can be optimized through routine experimentation.

Numerous types of tubular supports may be utilized with the illustrated embodiment. These supports may be self expandable or expandable via, for example, an internal expanding device such as a balloon. See e.g., U.S. Pat. No. 6,123,722, which is hereby incorporated by reference herein. In one embodiment, a self expandable support may be formed of a shape memory alloy that can be deformed from an original, heat-stable configuration to a second heat-unstable configuration. See e.g., U.S. Pat. No. 6,051,020, which is hereby incorporated by reference herein. Such supports may also be formed from a wire or a piece of metal tubing that is laser cut. In another embodiment, the support is formed from any of a variety of self-expandable tubular wire supports, such as the tubular wire supports disclosed in U.S. Pat. Nos. 5,683,448, 5,716,365, 6,051,020, 6,187,036, which are hereby incorporated by reference herein, and other self-expandable configurations known to those of skill in the art. In general the support may comprise a series of end to end segments, each segment comprising a zig-zag wire frame having a plurality of apexes at its axial ends, and wire struts extending therebetween. Opposing apexes of adjacent segments may be connected in some or all opposing apex pairs, depending upon the desired performance.

It should be appreciated that in modified embodiments the tubular support or skeleton may be positioned on only certain portions of the axial length of the prosthesis 42. For example, in one embodiment, only the distal and proximal ends 46A, 46B, 50A, 50B of the prosthesis are provided with a tubular skeleton or support. In other embodiments, the prosthesis 42 is fully supported by a tubular support. (i.e., the tubular support extends through the entire length of the prosthesis). In still other embodiments, the prosthesis 42 may be formed with out a tubular support. In such embodiments, distal and proximal ends 46A, 46B, 50A, 50B of the prosthesis preferably include tissue anchoring structures as described above.

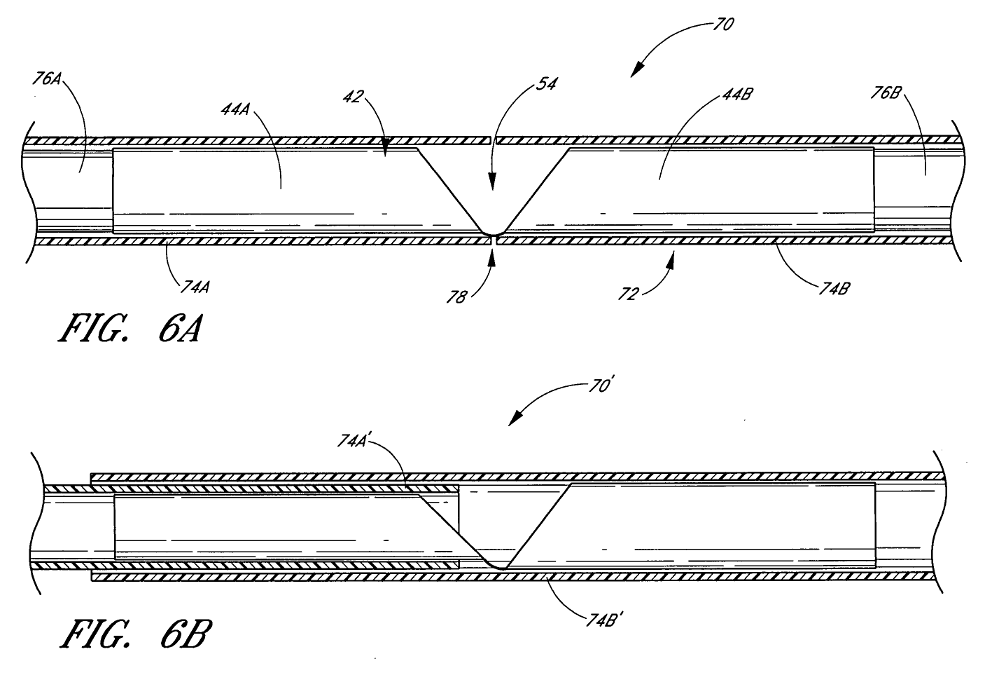

FIG. 5 illustrates one manner for forming the flexible connection 54 between the first and second tubes 44A, 44B of the prosthesis 42. As shown in FIG. 5, the prosthesis comprises a single outer tubular sheath 60 in which wire supports 62A, 62B are positioned. A slot or wedge shaped section 64 of the sheath 60 is removed from a portion of the sheath 60 that lies in a space between the two wire support sections 62A, 62B. This leaves a hinge strip 54 of the sheath 60 between the adjacent tubular supports 62A, 62B. The prosthesis 42 may be flexed about the flexible connection 54 by bending the ends of the prosthesis 42 in the directions of the arrows labeled A in FIG. 5 to configure the prosthesis as illustrated in FIG. 2. In this manner, the connecting hinge strip of the sheath 60 forms the flexible connection 54 between the legs of the prosthesis 42.

The wire supports 62A, 62B may also extend across or be connected across the flexible connection 54. In modified embodiments, other methods and devices may be used to link the first and second tubes 44A, 44B together. For example, the flexible connection 54 may be formed by interlocking wire structures which form a series of pivotable links. Adjacent apexes 51, 53 (FIG. 4) may be pivotably linked to each other by a separate loop of metal or suture to provide a hinge. Alternatively, the opposing apexes 51, 53 may be directly interlinked with each other, without a distinct loop. In other embodiments, the flexible connection 54 may be formed from a fabric hinge with or without mechanical interlinking, or other structures as will be apparent to those of skill in the art in view of the disclosure herein. In another embodiment, the wire supports 62A, 62B may extend integrally across the flexible connection 54.

FIG. 5A illustrates a modified embodiment of a prosthesis 42. In this embodiment, at the distal ends of the tubes 44A, 44B, the graft follows the pattern of the tubular support 60. Accordingly, because the illustrated tubular support 60 has a zig-zag pattern, the end of the graft also has a zig-zag pattern. The zig-zag graft pattern can be formed by removing a portion of the graft (e.g., by cutting the graft) or the graft can be preformed into the zig-zag pattern. Preferably, the graft follows the zig-zag pattern for at least one level of the zig-zag pattern of the tubular support 60. In other embodiments, the graft may follow only a portion of one level of the pattern. This arrangement advantageously reduces the space occupied by the prosthesis 42 within the deployment catheter, particularly, in the region of the flexible connection 54 which tends to be the largest (with respect to diameter) part of the prosthesis 42.

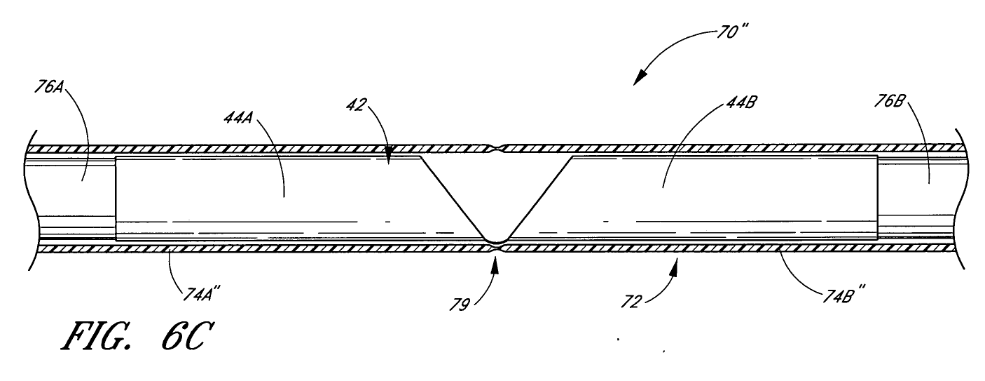

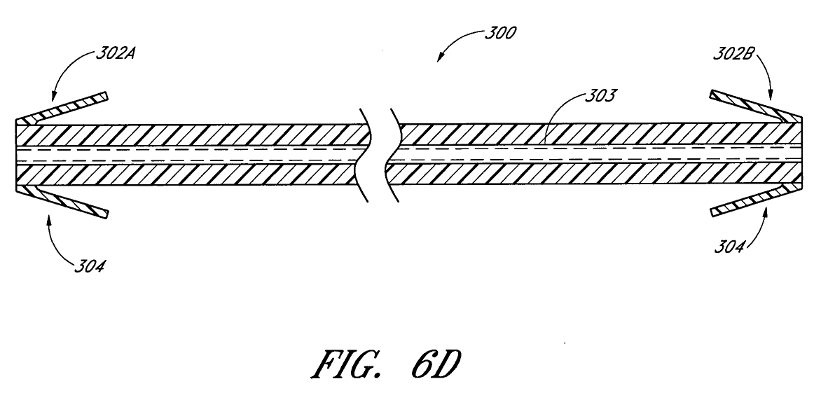

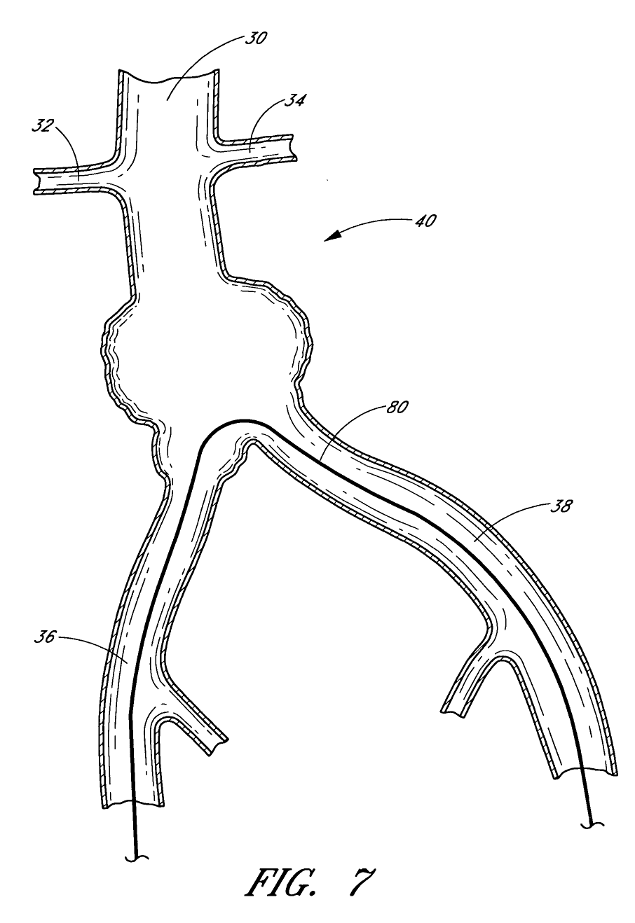

FIG. 6A is a partial cross-sectional side view of one embodiment of a deployment apparatus 70, which can be used to deploy the prosthesis 42 described above. The deployment apparatus 70 comprises an elongate flexible multicomponent tubular body 72 comprising a first (proximal) sheath 74A and second (distal) sheath 74B. Although not illustrated, an outer sheath may be positioned over the first and second sheaths 74A, 74B to span the junction 78 to enhance trackability during positioning as will be explained in more detail below.

The tubular body 72 and other components of this system can be manufactured in accordance with any of a variety of techniques well known in the catheter manufacturing field. Extrusion of tubular catheter body parts from material such as Polyethylene, PEBAX, PEEK, nylon and others is well understood. Suitable materials and dimensions can be readily selected taking into account the natural anatomical dimensions in the iliacs and aorta, together with the dimensions of the desired implant and percutaneous or other access site.

A pair of opposing stops or pushers 76A, 76B are axially movably positioned with respect to the sheaths 74A, 74B. The prosthesis 42 is positioned in a compressed or reduced diameter state within the sheaths 74A, 74B between opposing stops 76A, 76B. Preferably, the prosthesis 42 is mounted such that the link 54 is positioned generally at a junction 78 between the opposing ends of the sheaths 74A, 74B. As will be explained in detail below, proximal (inferior direction) retraction of the sheaths 74A, 74B through the respective iliac arteries and with respect to the proximal stops or pushers 76A, 76B, will deploy the prosthesis 42.

FIG. 6B is a partial cross-sectional side view a modified deployment apparatus 70′, which can be used to deploy the prosthesis 42 described above. In this embodiment, the first and second sheaths 74A′, 74B′ partially overlap each other. As such, the first sheath 74A′ has an outer diameter that is slightly smaller than the inner diameter of the second sheath 74B′. This arrangement advantageously eliminates the junction 78 between the first and second sheaths during translumenal navigation thereby eliminating or reducing the need for an outer sheath (not illustrated). The prosthesis 42 may be positioned with the flexible connection 54 within about 1 cm or 2 cm of the distal end of the first sheath 74A. Opposing stops (not illustrated) may be provided as described above.

FIG. 6C is a partial cross-sectional side view of another embodiment of a deployment apparatus 70″, which can be used to deploy the prosthesis 42 described herein. In this embodiment, the first and second sheaths 74A″, 74B″are detachably coupled to each other at their ends. For example, the illustrated first and second sheaths 74A″, 74B″ can be formed from a single tube that has a portion 79 that can be ruptured in a controlled manner. In such an embodiment, the portion 79 may comprise a weakened, a thinner and/or a serrated section of tubing. The portion 79 can also be pre-marked with incisions to facilitate separation. In other embodiments, the first and second sheaths 74A″, 74B″ can be separated in a controlled manner by providing a detachable link or linking mechanism between the two sheaths 74A″, 74B″. Any of a variety of links or linking mechanism may be used, such as, for example, various combinations of hoops, hooks and/latches and the like or fabric than can be un-knitted upon the pulling of a thread.

In the deployment devices of FIGS. 6A-6C, it may be advantageous to provide an inner tubular member 300 (see FIG. 6D), which is configured to extend through an inner lumen provided in the pushers 76A, 76B. The ends 302A, 302B of the tubular member 300 can be coupled to the ends of the pushers 76A, 76B. The sheaths 74A, 74B, in turn, are coupled to the pushers 76A, 76B. In this manner, both ends of the deployment apparatus are coupled together by the inner tubular member 300. In the illustrated embodiments, the inner member 300 is coupled to the pusher 76A, 76B by flexible sleeves 304 provided on the ends 302A, 302B. The sleeves 304 can be slip fitted over the ends of the pushers 76A, 76B to couple the two components together. Other coupling mechanisms can also be used, such as, for example, hooks, latches, etc. In addition, a simple slip-fit between the two components may sufficiently couple to the two components together. The inner tubular member 300 is preferably provided with a guidewire lumen 303 such that the apparatus can advanced over a guidewire. The inner tubular member 300 can be removed form the apparatus such that the two portions of the deployment apparatus can be separated from each other and/or moved independently from each other.

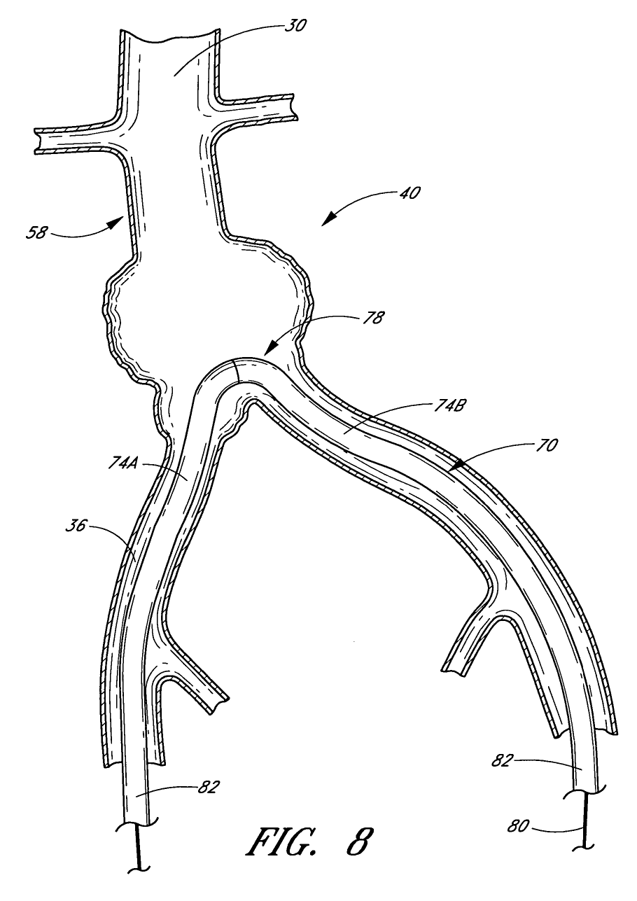

A technique for deploying the prosthesis 42 using the deployment apparatus 70 described in FIG. 6A will now be described with reference to FIGS. 7-11. With initial reference to FIG. 7, there is disclosed a schematic representation of the abdominal part of the aorta 30 and its principal branches as described above. A standard 0.035″ diameter guidewire 80 is in position across the ipsilateral and contralateral iliac arteries 36 and 38. The guidewire 80 may be introduced, for example, from the contralateral side through a percutaneous puncture, and advanced superiorly towards the aorta 30. A retrieval catheter (not shown) is introduced superiorly through a vascular access site and into the ipsilateral iliac, and used to grasp the guidewire 80 and retract it inferiorly and out through the ipsilateral vascular access site in accordance with known techniques.

As shown in FIG. 8, the deployment apparatus 70 is advanced over the guidewire 80 from, for example, the ipsalateral access site along the guidewire 80 and out the contralateral access site. The guidewire 80 can thereafter be removed. The opposing device proximal end 81 and device distal end 82 of the deployment apparatus 70 extend outside the patient on the ipsalateral iliac side and the contralateral iliac side. The junction 78 between the opposing ends of the sheaths 74A, 74B is preferably positioned between the right and left common iliac arteries 36, 38. The catheter is rotationally oriented such that the flexible connection 54 is on the inferior side. To aid positioning, one or both of the opposing ends of the outer sheaths 74A, 74B may be provided with radio opaque markers in the vicinity of the junction 78 to enable visualization during placement. Any of a variety of techniques may be used to provide radio opaque markers, such as, for example, providing the outer sheaths with bands or staples made of radio opaque material or dispersing radio opaque material into the material that forms the sheaths.

Although not illustrated, the deployment apparatus 70 may be advanced over the guidewire with the outer sheath (not illustrated) positioned over the first and second sheaths 74A, 74B and spanning the junction 78. Once the junction is properly positioned approximately mid-bifurcation, the outer sheath may be removed to expose the junction 78.

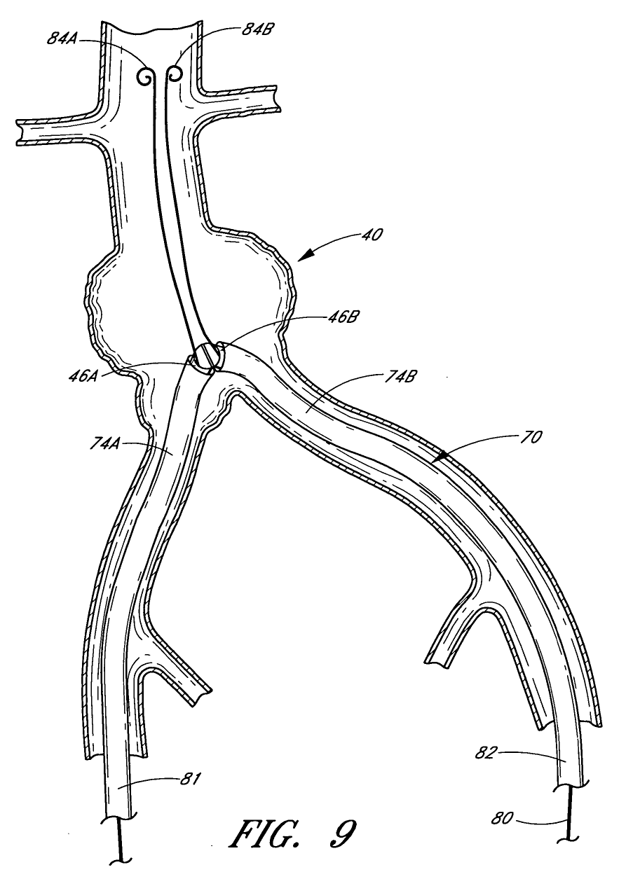

As shown in FIG. 9, the outer sheaths 74A, 74B may then be partially inferiorly retracted to expose the opposing ends 46A, 46B of the prosthesis 42. First and second guide wires 84A, 84B can be advanced through the tubes 44A, 44B of the prosthesis 42, one from the contralateral side and one from the ipsilateral side, until the distal ends of the guidewires 84A, 84B exit the deployment apparatus 70 through the junction 78 between opposing ends of the outer sheaths 74A, 74B. The guidewires (or single guidewire, if desired) may then be navigated across the aneurysm 40 into the aorta 30. With the deployment apparatus 72′ of FIG. 6A, the second sheath 74B′ may be partially withdrawn inferiorly with respect to the first sheath 74A′ so as to provide a gap 78 between the first and second sheaths 74A′, 74B′ through which the guidewires may be advanced as described above.

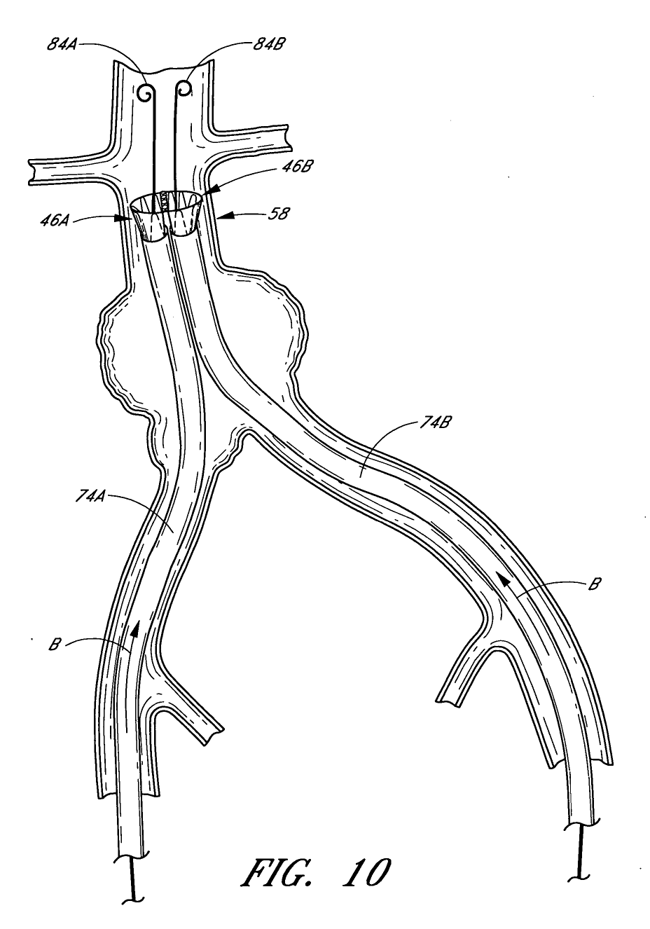

The opposing superior ends 46A, 46B of the prosthesis 42 are then positioned at the aortic neck 58 by pushing the proximal end 81 and the distal end 82 of the deployment apparatus 70 extending out of the patient from the ipsilateral and contralateral access sites in the superior direction as illustrated by the arrows labeled B in FIG. 10. In response, the two tubes 44A, 44B of the prosthesis 72 pivot about the flexible connector 54 and the deployment apparatus 70 can be used to push the opposing ends 46A and 46B of the prosthesis 42 over the guidewires 84A, 84B and into position as shown in FIG. 10. The opposing ends of the first and second sheaths 74A, 74B, in the vicinity of the junction 78, may contact and push against the flexible connection 54 during advancement of the prosthesis 42 across the aneurysm. To aid visualization during positioning, the superior ends 46A, 46B of the prosthesis 42 and/or sheaths 74A, 74B may be provided with radio opaque markers to enable visualization during placement. Any of a variety of techniques may be used to provide such radio opaque markers, such as, for example, providing the sheaths with bands or staples made of radio opaque material or dispersing radio opaque material into or onto the sheath material or onto the tubular support, or crimping, welding or otherwise attaching markers to the wire support.

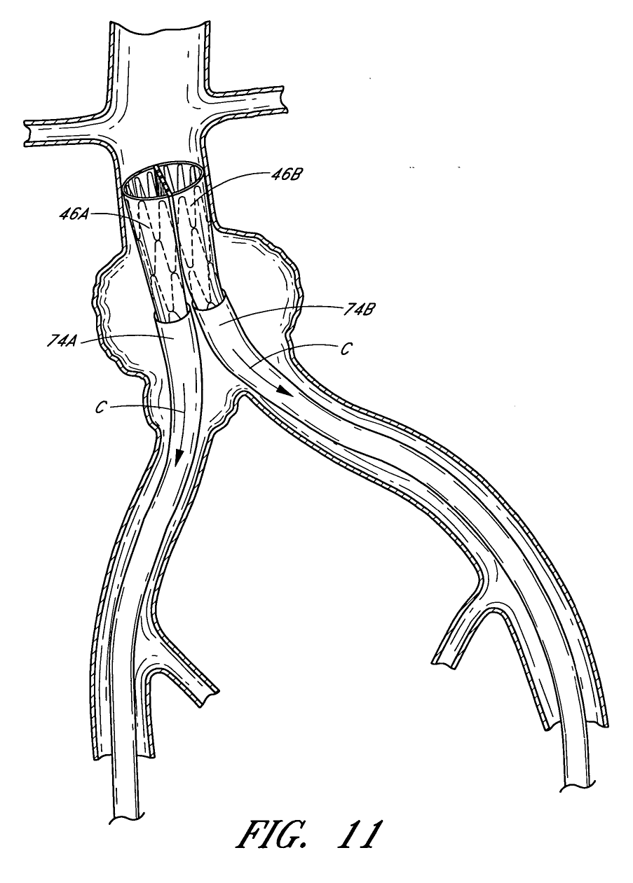

As shown in FIG. 11, the first and second sheaths 74A, 74B can then be inferiorly withdrawn in the direction of the arrows marked “C” while the stops 76A, 76B are held axially stationary to deploy the ends 46A, 46B of the prosthesis 42 as shown in FIG. 11. This allows the superior end of the implant to self expand within the aorta. Continued proximal retraction of the first and second sheaths 74A, 74B deploys the inferior ends 50A, 50B of the prosthesis 72 in the right and left common iliac arteries 36, 38 as shown in FIG. 1. The deployment catheter 70 may thereafter be proximally withdrawn from the patient by way of the first and second percutaneous access sites.

As mentioned above, it is sometimes desirable to extend the prosthesis over or beyond the renal arteries so as to maximize the overlap between graft material and the healthy infrarenal aortic wall 58 and thereby promote a good seal within the artery. Such an arrangement is particularly advantageous if the aneurysm is positioned near the renal arteries.

FIGS. 12-14 illustrate an exemplary embodiment of a prosthesis 100 particularly configured such that it may be extended over and/or beyond the renal arteries 32, 34. This exemplary embodiment is generally configured similar to the prosthesis 42 described above. Accordingly, reference numbers used above will be used to describe similar components.

As with the previous embodiment, the prosthesis 100 comprises a first tubular member or tube 44A and a second tubular member or tube 44B. The first tubular member 44A has a device distal end 46A, which defines a device distal opening (not shown), and a device proximal end 50A, which defines a proximal opening (not shown). In a similar manner, the second tubular member 44B has a device proximal end 46B, which defines a proximal opening (not shown), and a device distal end 50B, which defines a distal opening (not shown). The distal end 46A and proximal end 46B of the tubes 44A, 44B are connected together by a flexible connection or hinge 54 as described above. The tubes 44A, 44B may be formed in a variety of manners including a combination of tubular support or skeleton and a sleeve. In the illustrated embodiment, the tubes 44A, 44B are formed from a wire support 62A, 62B and a tubular sheath 60, which in the illustrated embodiment is generally positioned over the wire support 62A, 62B.

As shown in FIG. 12, the prosthesis 54 may be positioned such that the hinge 54 is positioned at or above the renal arteries 32, 34. Accordingly, the distal end 46A of the first tubular member 44A and the proximal end 46B of the second tubular member 44B extend over and/or beyond the renal arteries 32, 34. To permit blood flow from the renal arties 32, 34, the portions of the distal end 46A of the first tubular member 44A and the proximal end 46B of the second tubular member 44B that extend over the renal arteries 32, 34 are not covered with the tubular sheath 60. In this manner, blood from the renal arties 32, 34 may flow through the exposed wire supports 62A, 62B while the wire supports 62A, 62B contact the arterial wall to provide support for the prosthesis 100. In other embodiments, the wire supports 62A, 62B may be provided any of variety of tissue anchoring structures as described above.

In the illustrated arrangements, the wire supports 62A, 62B are exposed by cutting or forming an edge 102A, 102B (see FIG. 13) which extends from to the outer sides of the prosthesis 100 distally to the hinge 54 or inner side of the tubular members 44A. The illustrated edge 102A, 102B is straight, however, in modified embodiments, the edge 102A, 102B may be curved, segmented etc. Other arrangements for allowing blood from the renal arties 32, 34 to pass through the prosthesis 100 may also be used. For example, the porosity of the sleeve 60 in the proximal region may be increased and/or various holes or openings may be formed in the sleeve 60. This arrangement also advantageously reduces the space occupied by the prosthesis 100 within the deployment catheter, particularly, in the region of the flexible connection 54 which tends to be the largest (with respect to diameter) part of the prosthesis.

With continued reference to FIGS. 12 and 13, in this embodiment, the tubes 44A, 44B of the prosthesis 100 terminate within the aneurysm 40. Accordingly, leg extensions 104A, 104B may be attached to the prosthesis such that the assembled prosthesis 100 extends across the aneurysm. The extensions 104A, 104B may be formed in a variety of manners and may include a skeleton and sleeve as described above. Various attachment devices (e.g., barbs, hooks, etc.) may be provided to facilitate attachment of the extensions 104A, 104B to the tubes 44A, 44B. For example, in the illustrated embodiment, a portion 106A, 106B, of the tubular support wire support 62A, 62B is folded over the sleeve 60 such that it lies on the outside of the sleeve 60.

The extensions 104A, 104B may be attached in situ (see e.g., U.S. Pat. No. 6,685,736, the disclosure of which is hereby incorporated by reference in its entirety herein) or before deployment. In certain embodiments, the extensions 104A, 104B may comprise self expandable grafts which are inserted into and expanded within the tubes 44A, 44B. See e.g., (U.S. Pat. No. 6,685,736, the disclosure of which is hereby incorporated by reference in its entirety herein). Of course, the tubes 44A, 44B may also be configured to extend across the aneurysm. In such an embodiment, the portions 106A, 106B may over time become embedded in cell growth on the interior surface of the vessel thereby advantageously resisting migration and reducing leakage around the ends of the prosthesis 100.

FIGS. 14A and 14B illustrate another exemplary embodiment of a prosthesis 200. In this embodiment, the distal end 46A and proximal end 46B of the tubes 44A, 44B may be connected together in situ by a connection component 202 positioned generally at the distal end 46A and proximal end 46B of the tubes 44A, 44B. In the illustrated embodiment, the connection component 202 comprises a first member 204 and a second member 206 positioned on the distal end 46A and proximal end 46B of tubes 44A, 44B respectively.

In one arrangement, one or both of the members 204, 206 are formed of or have incorporated therein a material capable of producing a magnetic field that acts to maintain the members 204, 206 in a desired positional relationship. The magnetic field results in the connection component 202 maintaining the tubes 44A, 44B is a desired position and a preferably also provides a sealed interface between the openings 48A, 48B of the tubes 44A, 44B. The material used to form one or both of the members 204, 206 may be formed from any of a variety of suitable materials, such as, for example, magnetic, ferromagnetic or electromagnetic materials. Examples may also include rare earth and other high strength type magnets, such as, for example, NdFeB (Neodymium Iron Boron), SmCo (Samarium Cobalt), and Alnico (Aluminum Nickel Cobalt).

Those of skill in the art will appreciate that the amount of force exerted will depend on various factors including the materials used, the size of the magnets and the number of magnets. In addition, it may be advantageous to distribute the members 202, 204 generally across the interface between the tubes 44A, 44B to promote a sealed interface. Sealing members 208a,b may be provided along side and/or between the connection members 204, 206 to provide a sealing arrangement between the interface of the openings 48A, 48B of the tubes 44A, 44B. The sealing members 208a,b may be formed from any of a variety of deformable or electrometric materials. In such arrangements, the magnetic force brings the sealing members 208a,b towards each other to form a seal.

In the illustrated arrangement, the sealing members and/or the connection members 202, 204 are elongated members that extend generally across the interface of the openings 48A, 48B. In addition, the connection members 202, 204 are shows as being coupled to the exterior of the tubes 44A, 44B. However, in modified embodiments, the shape and number of connection members 202, 204 may be modified. In addition, the position of the connection members 202, 204 on the prosthesis may be modified. For example, it is anticipated that the connection members 202, 204 the may be integrally formed with the tubes 44A, 44B and our coupled to the interior of the tubes 44A, 44B.

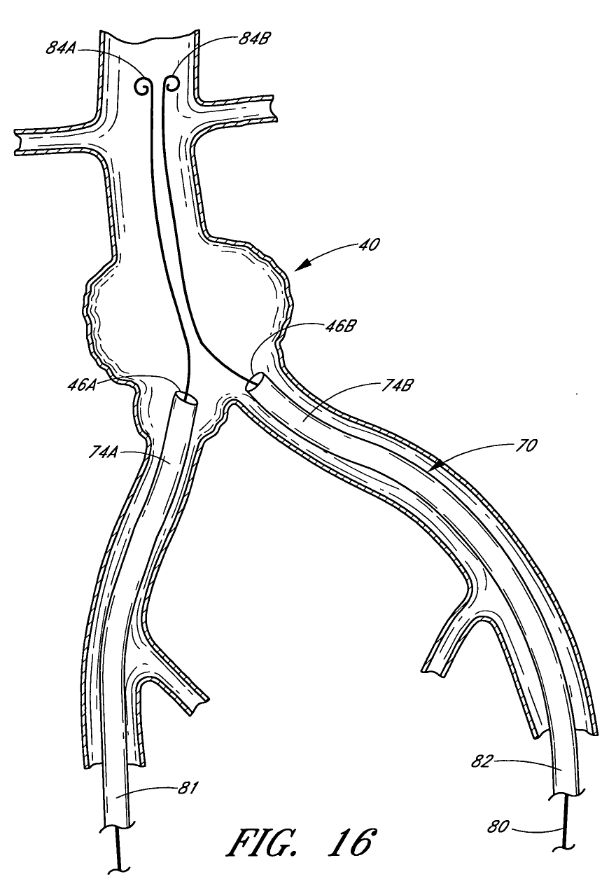

With reference to FIG. 15, a mechanical linkage 210 may be used in combination with the connection component 202 described above. In one embodiment, the magnetic force is used to form and/or promote the mechanical linkage 202 between the tubes 44A, 44B. The mechanical linkage may be formed from any of a variety of manners including various combinations of prongs, interlocking members, keys, latches, cams, interference fits and/or friction fits. In the illustrated embodiment, the mechanical linkage comprises a slot 212 with a narrowing neck 214 and a deflectable prong 216. The slot 212 is positioned on one tube 44A, 44B generally facing the prong 216 on the opposite tube. When the end 46A and proximal end 46B of the tubes 44A, 44B are brought together, the magnetic force draws the slot into the prong providing a mechanical linkage between the tubes 44A, 44B.

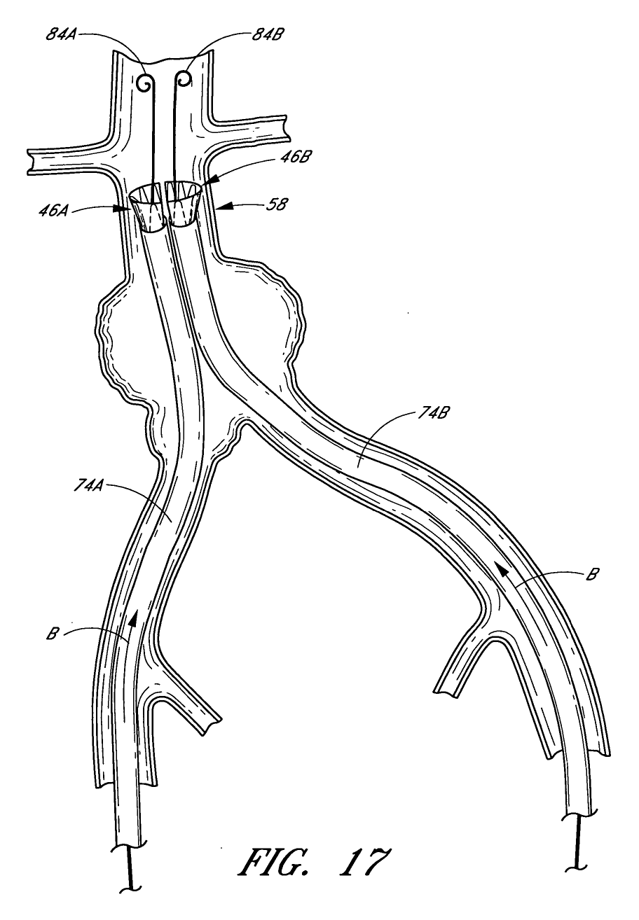

FIGS. 16-18 illustrate a technique for deploying the prosthesis 200 using the deployment apparatus 70 described above. With initial reference to FIG. 16, first and second guide wires 84A, 84B can be advanced through the ipsilateral and contralateral iliac arteries 36, 38 and across the aneurysm 40 into the aorta 30.

In this embodiment, because the first and second tubes 44A, 44B are not coupled to each other. Therefore, the first and second sheaths 74A, 74B may be introduced through an ipsilateral access site and a contralateral access site respectively and advanced independently through the ipsilateral iliac the ipsilateral and contralateral iliac arteries 36, 38 and across the aneurysm 40 into the aorta 30. As shown in FIG. 17, the opposing superior ends 46A, 46B of the prosthesis 42 may be positioned generally at the aortic neck 58.

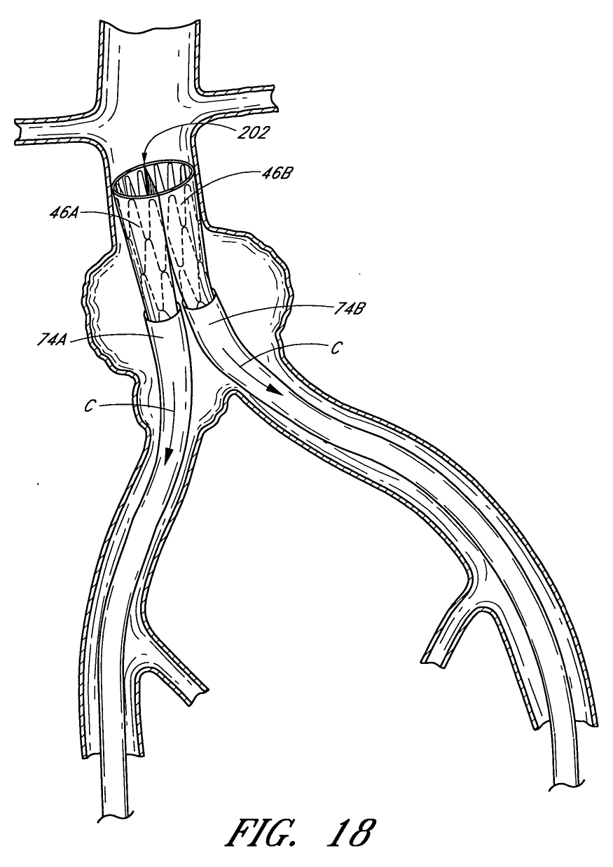

As shown in FIG. 18, the first and second sheaths 74A, 74B can then be inferiorly withdrawn in the direction of the arrows marked “C” while the stops 76A, 76B (not shown in FIG. 18) are held axially stationary to deploy the ends 46A, 46B of the prosthesis 42. This allows the superior end of the implant to self expand within the aorta. As the first and second sheaths 74A, 74B are inferiorly withdrawn, the ends 46A, 46B of the prosthesis are drawn together by the connection component 202. As explained above, a magnetic field acts to maintain the members 204, 206 and therefore the tubes 44A, 44B, in a desired positional relationship. In addition, the prosthesis 200 may include a mechanical linkage 210 (described above) to further secure the tubes 44A, 44B together.

Continued proximal retraction of the first and second sheaths 74A, 74B deploys the inferior ends 50A, 50B of the prosthesis 72 in the right and left common iliac arteries 36, 38 . The deployment catheter 70 may thereafter be proximally withdrawn from the patient by way of the first and second percutaneous access sites. In some embodiments, it may be advantageous to provide additional fasteners (e.g., staples, sutures, etc.) between the superior ends of the tubes 44A, 44B. These fasteners may be applied before or after the catheter 70 is removed.

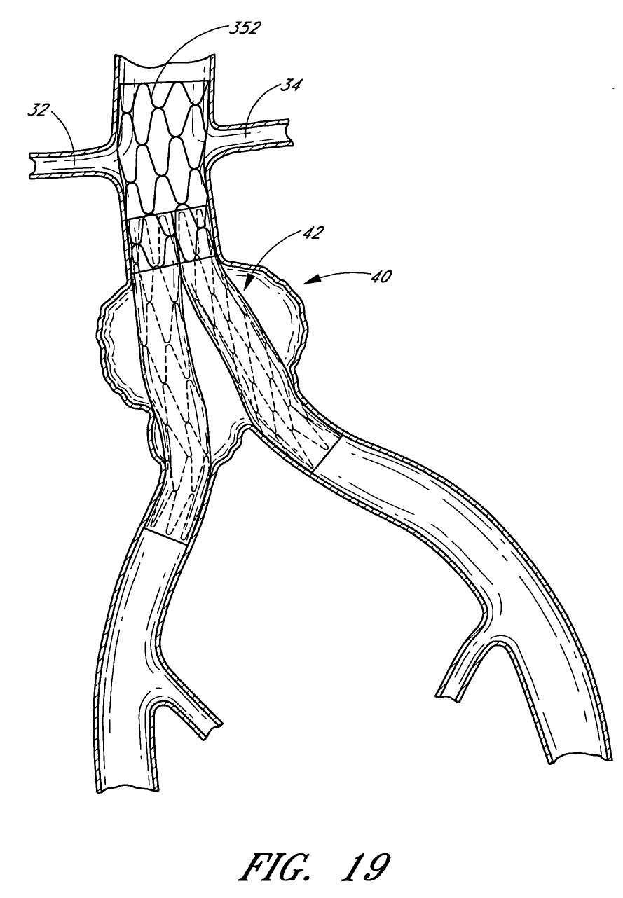

FIG. 19 illustrates an embodiment in which the prosthesis 42 is used in combination with a secondary prosthesis 350 that is placed at the proximal (with respect to blood flow) end of the prosthesis 42 to achieve enhanced anchoring. In this embodiment, the secondary prosthesis 350 preferably comprises an uncovered tubular support 352 such that the prosthesis 350 can extend over the renal arteries 32, 34 without significantly restricting blood flow. In other embodiments, the prosthesis 350 can comprise a sleeve that partially or wholly covers the tubular support. In such embodiments, the sleeve may be provided with one or more openings to permit blood flow through the walls of the tubular support 352.

The tubular support 352 may be self expandable or expandable via, for example, an internal expanding device such as a balloon. In another embodiment, a self expandable support may be formed of a shape memory alloy that can be deformed from an original, heat-stable configuration to a second heat-unstable configuration In the illustrated embodiment, the secondary prosthesis 352 comprises a series of end to end segments, each segment comprising a zig-zag wire frame having a plurality of apexes at its axial ends, and wire struts extending therebetween. Opposing apexes of adjacent segments may be connected in some or all opposing apex pairs, depending upon the desired performance. The secondary stent can include any of a variety of tissue anchoring structures (not shown), such as, for example, barbs, and/or hooks to enhance attachment to the native tissue and resist migration of the prosthesis 350.

As shown in FIG. 19, the secondary prosthesis 350 can be placed at the level of the renal arteries 32, 34 and can even extend above the renal arteries. By using a longer contact area with the anatomy, the secondary prosthesis 350 can achieve very strong anchoring with the anatomy. The distal end of the prosthesis 42 can then be expanded, at least partially, within the secondary stent, preferably, below the renal arteries 32, 34. Anchoring between the prosthesis 42 and the secondary stent 350 can be enhanced by providing anchoring structures, such as, for example, barbs, and/or hooks between the secondary prosthesis 350 and the prosthesis 42. In another embodiment, a polymer and/or another adhesive material can be placed between the two devices 350, 42 to enhance anchoring and minimize or eliminate leaks between the two prosthesis. In other embodiments, staples or sutures can be used to couple to the devices 350, 42 together.

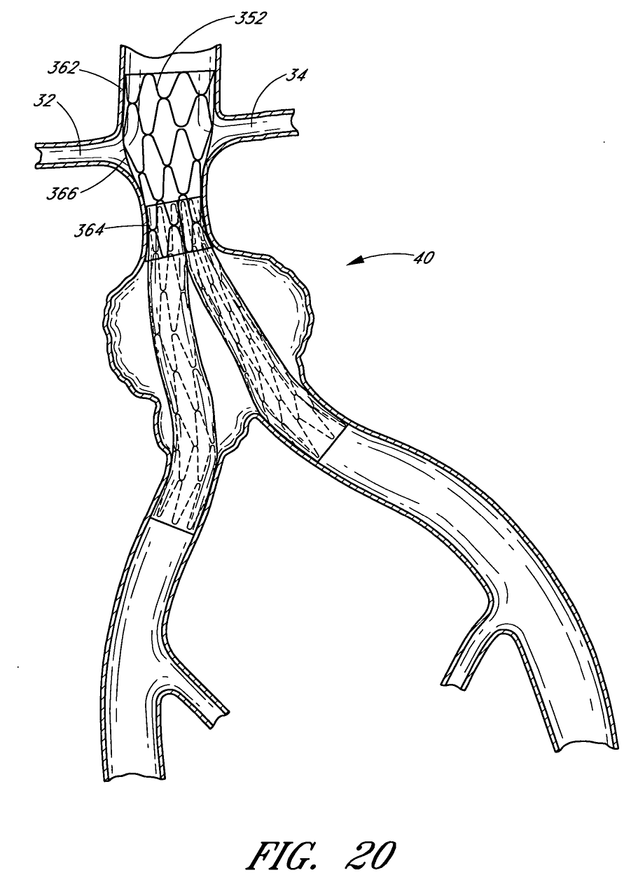

FIG. 20 illustrates a modified embodiment of the embodiment of FIG. 19 in which the secondary prosthesis 360 is configured to allow for a size difference between the local anatomy and the prosthesis 42. As shown, the diameter of the anatomy proximal (with respect to blood flow) to the aneurysm 40 is larger than the diameter of the prosthesis 42 Accordingly, the secondary prosthesis 360 is provided with a device distal portion 362 that has a larger diameter than a device proximal portion 364 of the prosthesis 362. A transition region 366 lies between the distal and proximal portions 362, 364. In other aspects, the stent 360 can be configured as described above. The distal end 362 of the prosthesis 360 is positioned within the larger diameter anatomy which, in the illustrated embodiment, lies proximal (with respect to blood flow) of the aneurysm 40 and renal arteries 32, 34, while the distal end of the prosthesis 42 is positioned within the smaller diameter proximal end 364 of the prosthesis 362. This allows a smaller diameter prosthesis 42 and, thus, a smaller diameter deployment catheter to be used to treat a larger vessel.

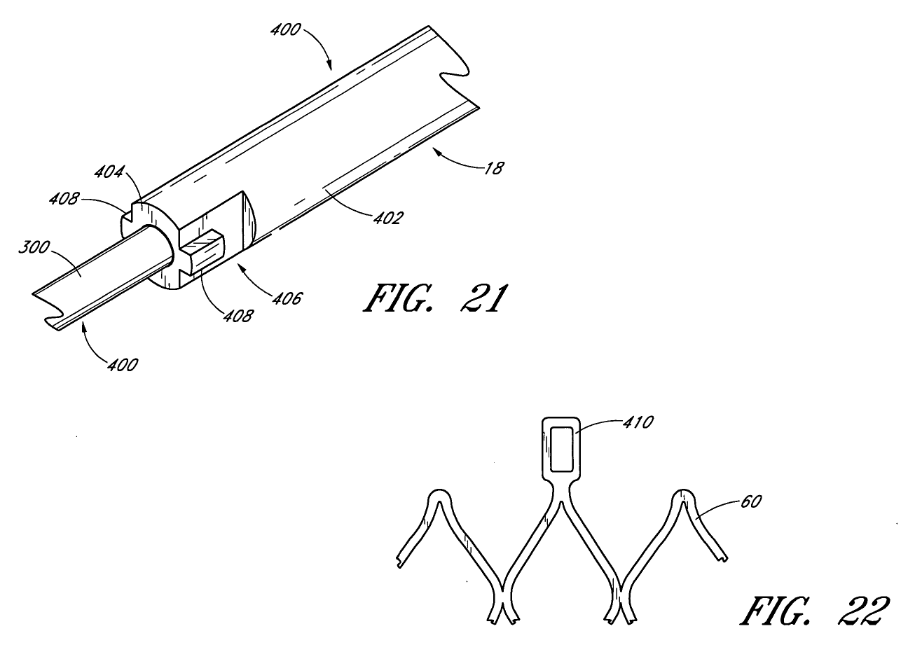

FIG. 21 is a side perspective view of an embodiment of a pusher 400 that can be used in combination with the deployment devices described above with reference to FIGS. 6A-6C. In this embodiment, the pusher 400 includes an elongated proximal portion. The proximal portion defines a distal facing pushing surface 404, which can be used to push the prosthesis 42 out of a sheath 74A, 74B as described above. An inner tubular member 300 (as described above) can extend through an inner lumen (not shown) that extends through the pusher 300. The inner tubular member 300 preferably defines a guidewire lumen (not shown) such that the pusher 400 can be advanced over a guidewire.

The distal end 406 of the pusher 400 includes a pair of protrusions or notches 408. The protrusions or notches 408 are configured to be inserted within corresponding attachment loops or openings (see FIG. 22), which can be formed at the proximal ends of the tubular support structure 60 of the prosthesis 42. The loops 410 and protrusions 408 are used in combination to couple the prosthesis 42 to the pusher 400. In this manner, a leg of the prosthesis 42 can be compressed and positioned in a sheath 74A, 74B by coupling the loops 410 and protrusions 408 to each other and proximally retracting the pusher 400 into a sheath 74A, 74B. During deployment of the prosthesis 42, the pusher 400 is advanced through the sheath to push the distal end of the prosthesis 42 out of the sheath. As the sheath expands, the loop 410 disengages from the protrusion 408 thereby releasing the prosthesis 42 from the pusher 400. Those of skill in the art will recognize that various other mechanisms may be used to couple the pusher 400 to the prosthesis 42 including, but not limited to, various combinations of hooks, barbs, prongs, and the like. It should also be appreciated that a pair of pushers 400 would be used in the deployment apparatus, one for each leg of the prosthesis.

While a number of preferred embodiments of the invention and variations thereof have been described in detail, other modifications and methods of using and medical applications for the same will be apparent to those of skill in the art. Accordingly, it should be understood that various applications, modifications, combinations, sub-combinations and substitutions may be made of equivalents without departing from the spirit of the invention or the scope of the claims.

What is claimed is:

1. A expandable bifurcation graft, comprising:
a first tubular body, having a superior end and an inferior end;
a second tubular body, having a superior end and an inferior end; and
a magnetic connection between the superior end of the first tubular body and the superior end of the second tubular body;
wherein the superior ends of the first and second tubular bodies are configured such that when the tubular bodies are connected by the magnetic connection about into a side by side relationship, each of the superior ends defines a semi circular opening.

2. A expandable bifurcation graft as in claim 1, wherein the bifurcation graft comprises a self expandable wire frame..

3. An vascular prosthesis comprising:
a first tubular segment having a device distal end and a device proximal end, the distal end defining a distal opening and the proximal end defining a proximal opening;
a second tubular segment also having a device distal end and a device proximal end, the distal end defining a distal opening and the proximal end defining a proximal opening; and
a magnetic link for connecting the distal ends of the first and second tubular segments.

4. The vascular prosthesis of claim 3, wherein the distal openings of the first and second tubular segments are D-shaped with one straight side and the magnetic link is disposed between the straight sides of the first and second tubular segments.

5. The vascular prosthesis of claim 3, wherein the first tubular segment and the second tubular segment comprise a tubular support and a sleeve.

6. The vascular prosthesis of claim 5, wherein at least a portion of the tubular support is exposed at the distal ends of the first and second tubular segments.

7. A method of deploying a vascular prosthesis comprising:
providing a deployment apparatus comprising an first outer sheath having a device distal end and a device proximal end and a second outer sheath also having a device distal end and a device proximal end;
providing a vascular prosthesis comprising first and second tubular segments, the first tubular segment positioned within the first outer sheath and the second tubular segment positioned in the second outer sheath;
advancing the first and second outer sheaths independently through an ipsilateral ialac artery and contralateral iliac artery in a distal direction until the distal ends of the first and second outer sheaths are positioned at an aortic neck; and
proximally retracting the first and second outer sheaths to deploy the prosthesis and allow the distal ends of the first and second tubular segments to attach to each other.

8. A method of deploying a vascular prosthesis as in claim 7, wherein, when the prosthesis is deployed, the distal end of the first and second tubular segments are attached via a magnet force.

9. A method of deploying a vascular prosthesis as in claim 8, wherein, when the prosthesis is deployed, the distal end of the first and second tubular segments are attached via a mechanical connection.

10. A method of treating an aneurysm near bifurcation of a vessel into a first branch and a second branch, comprising the steps of:
deploying a first prosthesis at a position proximal, with respect to blood flow, to the aneurysm;
providing a catheter having a device proximal portion, a device distal portion, and a deployment zone therebetween;
positioning the catheter such that the device proximal portion extends into the first branch, the device distal portion extends into the second branch, and the deployment zone is aligned with the vessel;
advancing the deployment zone superiorly into the vessel; and
deploying a bifurcation graft from the catheter such that a device distal end of the bifurcated graft is positioned within the first prosthesis.

11. The method of claim 10, further comprising coupling the bifurcated prosthesis to the first prosthesis.

12. The method of claim 11, wherein coupling the bifurcated prosthesis to the first prosthesis comprises at least one of applying an adhesive, using hooks, using hooks or using barbs.

13. The method of claim 10, wherein the step of deploying a first prosthesis at position proximal, with respect to blood flow, to the aneurysm comprises deploying the first prosthesis such that it spans, at least partially, over a branch vessel that is proximal, with respect to blood flow, of the aneurysm.

14. The method of claim 10, wherein the first prosthesis has a device distal portion and a device proximal portion, the device distal portion having a larger diameter than the device proximal portion.

15. A vascular prosthesis assembly comprising:
a first prosthesis comprising a tubular structure; and
a second prosthesis comprising
a first tubular segment having a device distal end and a device proximal end, the distal end defining a distal opening and the proximal end defining a proximal opening;
a second tubular segment also having a device distal end and a device proximal end, the distal end defining a distal opening and the proximal end defining a proximal opening; and
a flexible link for connecting the distal ends of the first and second tubular segments;
wherein the device distal ends of the first and second tubular segments are configured to be expanded within the first prosthesis.

16. A vascular prosthesis assembly as in claim 15, wherein the first prosthesis has a device distal portion and a device proximal portion, the device distal portion having a larger diameter than the device proximal portion.

17. A vascular prosthesis assembly as in claim 15, wherein the first prosthesis comprises an uncovered tubular support structure.

18. A vascular prosthesis assembly as in claim 15, wherein the first prosthesis comprises a tubular support that is at least partially covered by a sleeve.

19. A vascular prosthesis as assembly in claim 15, wherein the first prosthesis comprises an anchoring mechanism configured to enhance anchoring between the first prosthesis and a patient's anatomy. .

20. A vascular prosthesis assembly as in claim 15, wherein an anchoring mechanism is provided between the first prosthesis and the second prosthesis.

21. A vascular prosthesis assembly as in claim 15, further comprising an adhesive material between between the first prosthesis and the second prosthesis

22. An vascular prosthesis comprising:
a first tubular segment having a device distal end and a device proximal end, the distal end defining a distal opening and the proximal end defining a proximal opening, the first tubular segment comprising a polymeric sleeve and a support structure comprising a series of end to end segments, each segment comprising a zig-zag frame;
a second tubular segment also having a device distal end and a device proximal end, the distal end defining a distal opening and the proximal end defining a proximal opening; the second tubular segment comprising a polymeric sleeve and a support structure comprising a series of end to end segments, each segment comprising a zig-zag frame; and
a flexible link for connecting the distal ends of the first and second tubular segments;
wherein at the device distal ends of the first and second tubular segments, the polymeric sleeves have a zig-zag edge that follows, at least partially, the zig-zag frame.

23. A vascular prosthesis assembly, comprising:
a deployment apparatus comprising a first outer sheath having a device distal end and a device proximal end and a second outer sheath also having a device distal end and a device proximal end;
a vascular prosthesis positioned within the first and second outer sheaths; the vascular prosthesis comprising first and second tubular segments that are connected together at their distal ends by a link;
a first pusher positioned within the first outer sheath between a device distal end of the first outer sheath and a first tubular segment of the vascular prosthesis; and
a second pusher positioned within the second outer sheath between a device proximal end of the second outer sheath and a second tubular segment of the vascular prosthesis.

24. A vascular prosthesis assembly as in claim 23, wherein the first tubular segment and the second tubular segment are detachable coupled to the first and second pushers respectively.

25. A vascular prosthesis assembly as in claim 23, wherein the first tubular segment and the second tubular segment are detachably coupled to the first and second pushers by a mechanism.

26. A vascular prosthesis assembly as in claim 25, wherein the mechanism comprises a loop on a support structure of the first and second tubular segment and a protrusion on the first and second pushers.

27. A vascular prosthesis assembly as in claim 23, further comprising an inner member that extend through a lumen that extend through the first and second pushers, the inner member defining a guidewire lumen.

28. A vascular prosthesis assembly as in claim 27, wherein the inner member is detachably coupled to the first and second pushers.

29. A vascular prosthesis assembly as in claim 28, wherein the first and second outer sheaths are detachably coupled to the first and second pushers respectively.

30. A vascular prosthesis assembly as in claim 23, wherein the distal end of the first outer sheath and the proximal end of the second outer sheath are detachably coupled to each other.

31. A vascular prosthesis assembly as in claim 23, wherein the distal end of the first outer sheath and the proximal end of the second outer sheath configured to be detached from each other by rupturing a portion of tubing.
